(19) United States
(12) Patent Application Publication
(10) Pub. No.: US 2013/0317420 A1
(43) Pub. Date: Nov. 28, 2013

(54) DEVICE AND METHOD FOR ENTERING USER INFORMATION INTO MEDICAL DEVICES

(71) Applicant: Fresenius Medical Care Deutschland GmbH, (US)

(72) Inventor: Wolfgang WEHMEYER, Tuebingen (DE)

(73) Assignee: Fresenius Medical Care Deutschland GmbH, Bad Homburg (DE)

(21) Appl. No.: 13/891,560

(22) Filed: May 10, 2013

Related U.S. Application Data
(60) Provisional application No. 61/646,512, filed on May 14, 2012.

(30) Foreign Application Priority Data
May 14, 2012 (DE) 102012009517.1

(51) Int. Cl. A61M 1/30; A61M 1/28

(52) U.S. Cl. CPC A61M 1/30; A61M 1/28
USPC 604/29; 700/83

(57) ABSTRACT

The present invention relates to the field of a device and a method for entering user information into medical devices. It is based on the problem of supporting the work of the attending medical personnel in the operation of medical devices and to ensure that unsuitable settings are not made inadvertently or accidentally for the concrete treatment situation in the operation of such medical devices. For this purpose a method and a device are proposed in which a second parameter value characterizing the medical device, entered into an input device is verified and depending on this verification it is ordered that the first or second parameter value or neither the first nor the second parameter value can be or is accepted into a control unit which controls the machine.

### TECHNICAL FIELD

The invention relates to the field of devices and methods for entering user information into medical devices.

### STATE OF THE ART

Medical devices are understood in particular to also include fluid treatment devices such as blood treatment machines, in which a fluid from a patient is passed through a fluid line to a fluid treatment component by means of which the fluid treatment component is treated and can be returned back to the patient through the fluid line, which can be divided into an arterial branch and a venous branch. Examples of such blood treatment machines include hemodialysis machines in particular. One such blood treatment machine is the subject of DE 198 49 787 C1 from the present applicant.

Dialysis is a method of purifying the blood of patients with acute or chronic renal insufficiency. A fundamental distinction is made here between methods having an extracorporeal blood circulation, such as hemodialysis, hemofiltration or hemodiafiltration (hereinafter summarized under the term “hemodialysis”) and peritoneal dialysis, which does not have an extracorporeal blood circulation.

In hemodialysis, blood is passed through the blood chamber of a dialyzer, which is divided from a dialysis fluid chamber by a semipermeable membrane in an extracorporeal circulation. A dialysis fluid containing the blood electrolytes in a certain concentration flows through the dialysis fluid chamber. The substance concentration of the dialysis fluid corresponds to the concentration of the blood of a healthy person. During the treatment, the patient's blood and the dialysis fluid are passed by both sides of the membrane, usually in countercurrent at a predetermined flow rate. The substances that must be eliminated in urine diffuse from the blood chamber, through the membrane and into the chamber for dialysis fluid, while at the same time electrolytes present in the blood and in the dialysis fluid diffuse from the chamber with the higher concentration to the chamber with the lower concentration. If a pressure gradient is built up on the dialysis membrane from the blood side to the dialysate side by a pump, for example, which withdraws dialysate from the dialysate circuit downstream from the dialysis filter on the dialysate side, water is transferred from the patient's blood through the dialysis membrane into the dialysate circuit. This ultrafiltration process leads to the desired withdrawal of water from the patient's blood.

In hemofiltration, ultrafiltrate is withdrawn from the patient's blood by applying a transmembrane pressure in the dialyzer without passing the dialysis fluid by on the side of the membrane of the dialyzer opposite the patient's blood. In addition a sterile and pyrogen-free substituate solution can be added to the patient's blood. We speak of predilution or postdilution, depending on whether the substituate solution is added upstream or downstream from the dialyzer. The mass exchange takes place by convection in hemofiltration.

Hemodiafiltration combines the methods of hemodialysis and hemofiltration. A diffusive mass exchange between the patient blood and the dialysis fluid takes place through the semipermeable membrane of a dialyzer as well as filtering of plasma water through a pressure gradient on the membrane of the dialyzer.

Plasmapheresis is a method according to which the patient's blood is divided into the blood plasma and its corpuscular constituents (cells). The blood plasma that is separated is purified or replaced by a substituate solution and returned back to the patient.

In peritoneal dialysis, a patient's abdominal cavity is filled with a dialysis fluid which has a concentration gradient with respect to the endogenous fluids through a catheter which is guided through the abdominal wall. The toxins present in the body pass into the abdominal cavity through the peritoneum, which acts as a membrane. After a few hours, the dialysis fluid, which is now spent and is in the patient's abdominal cavity, is replaced. Water can be transferred from the patient's blood into the dialysis fluid by way of the abdominal wall through osmotic processes, thereby withdrawing it from the patient.

The methods for dialysis are usually performed with the help of automatic dialysis machines such as those distributed by the applicant under the brand name 5008 or sleep.safe, for example.

These dialysis machines are complex medical devices having extensive functions. Such machines are frequently operated by means of touchscreen displays, which are understood to be combined input and output devices.

Extensive operating steps and operator inputs into the dialysis machine are necessary for the preparation and treatment per se, these steps usually being performed by trained medical personnel.

For example, in the case of a hemodialysis treatment, the hemodialysis machine must be equipped with specific disposable parts (disposables) for the treatment and the patient. These include, for example, the blood tubing set and the dialysis filter.

In addition, various settings are to be made on the machine before or during the treatment. For example, the typical values for the flow rate of the blood pump or the dialysate flow rate must be set. Furthermore, relevant factors also include the dialysis time, the temperature of the blood in the extracorporeal blood circulation, the quantity of water to be withdrawn from the patient's blood, flow rates for the substituate solution, which is optionally mixed with the patient's blood, or concentration ratios for dilution of certain dialysis-specific concentrates, such as bicarbonate or sodium.

In addition, during the treatment certain situations may arise in which the dialysis machine outputs messages on an output device requiring an operator input, for example, a confirmation and/or acknowledgement or input of values by an operator. Such a message may be an alarm message, for example, which is displayed on a display screen, for example. However, the operator's attention can also be attracted by an optical signal device, for example, a lamp or an alarm sound.

It is important that an operator input into the dialysis machine must be made in order to process such messages.

Such operator entries are usually made on a touchscreen display.

The treatment is often performed in dialysis centers, where several dialysis machines are available for treating multiple patients simultaneously. The medical staff performs numerous treatments as part of their activity. This often necessitates repeatedly making the same settings on the dialysis machine for the treatment for different patients.

To offer some relief to the medical personnel in their work, it is possible to provide for the dialysis machine to automatically suggest standard values for various settings during preparation for the treatment by displaying these values on a touchscreen display, for example, so that medical personnel need only confirm these settings by an operator entry, for example, by operating an OK button on a touchscreen display.

During the treatment situations may also arise in which the dialysis machine requests an operator input, for example, when a monitoring device which is monitoring the treatment detects an alarm state.

For example, the incoming arterial blood pressure in the extracorporeal blood circulation may be monitored during a dialysis treatment. This blood pressure is measured on the intake side of the blood pump and has negative values with respect to the outside pressure. If the arterial blood pressure is too low, this may suggest that the blood flow rate is too high. Accordingly, an alarm message is output when a dialysis machine detects an arterial blood pressure that is lower than the limit value established previously.

At the same time, the dialysis machine may propose a reduction in the blood flow rate by a certain amount by prompting a corresponding output on a display. Then several possibilities for responding to the alarm message may be made available to the user via corresponding selection buttons, for example, as touch-sensitive surfaces on a touchscreen display. The operator may consent to the suggestion submitted by the dialysis machine by depressing a confirmation button or an OK button, for example, or the suggestion may be rejected by depressing a “no” button or a termination button.

There is the risk that the operator might perceive such requests for intentional confirmation of a suggestion submitted by the dialysis machine as an interfering interruption in a treatment sequence and might confirm the suggestion prematurely without consideration, even without determining the possible consequences of the suggestion. Therefore, this obstructs the protection of a change in the setting of the medical device, which is suggested automatically by the medical device.

With medical devices, in particular with dialysis machines, this could result in danger to the patient or could lead to suboptimal settings which could have a negative effect on the quality of the treatment.

Especially in everyday practice in dialysis clinics, there is sometimes a certain time pressure due the numerous treatments, which may result in acceptance and implementation of settings suggested by the dialysis machine prematurely and without consideration.

This problem does not occur only with dialysis machines but occurs in general with medical devices that make suggestions for settings via an output device, for example, a display screen or a touchscreen display, so that these settings can be selected by the operator only by brief confirmation, for example, by operating an OK button on the touchscreen display.

### DETAILED DESCRIPTION OF THE INVENTION

Consequently, the object of the invention is to create a device and a method such that, on the one hand, the work of the attending medical personnel is supported in the operation of medical devices, while on the other hand there is assurance that settings which are inappropriate for the specific treatment situation are not made carelessly or inadvertently in the operation of medical devices.

These problems are solved according to the invention by a method having the features of claim 1 and by a device having the features of claim 8.

Advantageous embodiments are the subject matter of the dependent claims.

The invention is thus based on the method for user input into a medical device, comprising the steps: output of a first parameter value characterizing the medical device or a message directed at the operator of the medical device, handwritten or oral input of a second parameter value characterizing the machine or an instruction, a verification of the second parameter value or instruction, and prompting, depending on this test, that the first or second parameter value or neither the first nor the second parameter value be accepted or acceptable for further processing in a control unit which controls the machine or that the instruction be transferred into a control unit which controls the machine.

In addition, the invention is based on a device consisting of a medical device with an input device, an output device and a control unit, which is equipped to verify an output, on an output machine, of a first parameter value characterizing the device or a message on directed at the operator of the medical device, and to verify a second parameter value characterizing the machine, said parameter value being input by handwriting or orally via the input device, or an instruction, and depending on the result of the verification, to accept or to make acceptable the first or the second parameter value or to accept or make acceptable neither the first nor the second parameter value, or to accept the instruction in a control unit controlling the machine.

A medical device is understood in particular to refer to a dialysis machine. The invention will be explained in greater detail below on the example of a machine equipped for hemodialysis. It will be clear to those skilled in the art that the invention can be used with any medical devices in which the problem may arise that suggestions made by the machine for settings on the respective machine which are not suitable for the specific treatment situation might be accepted inadvertently by the operator.

Such settings include parameter values for certain parameters which characterize the respective medical device. The corresponding parameter values include, for example, pump settings such as the flow rate, the dialysate flow rate or the substituate flow rate, but also values for the temperature of the extracorporeal blood circulation, the total volume of water that can be withdrawn from a patient's blood during a treatment or the duration of the treatment.

For other medical devices, these parameter values may each be applicable for other parameters which characterize and/or are specific for the respective machine. In the case of an infusion machine for example, the infusion rate and total volume of fluid to be infused are conceivable parameters.

The output device often includes a display which may also be embodied as touchscreen display. Such a touchscreen display combines an input device and an output device in one common surface in which it provides a touch-sensitive surface with which operator entries can be detected.

However, a spatial separation of the input a spatial separation of the input device and output device, for example, by a conventional display, for example, as a CRT monitor, LCD, plasma or OLED display is also conceivable, and/or as an output device and a touchpad which is separated spatially from the former and which provides as the input device a touch-sensitive surface with which the operators' entries can be detected.

It is also conceivable that the output device comprises a loudspeaker with which parameters and parameter values suggested by the medical device may be acoustically output as a medical device for example.

One such acoustic output device may also be present for an existing optical output device.

For entering operating information, a microphone may be provided. Parameter values spoken by the operator are then analyzed and processed further by a suitably programmed control unit having speech recognition software included.

Medical devices often have at least one control unit. One such control unit controls the components of the medical device on the basis of which parameter values which characterize the medical device and were made known to it among others. Such control units are often equipped with programmable microprocessors or microcontrollers such that the programs controlling them are filed in program data memories provided for this purpose. Programs pertaining to the settings of the machine can be parameterized by the operator entries. These operator entries thus include the parameter values which characterize the medical device.

Multiple control units are often provided in one medical device. In the sense of the present invention, a control unit is also understood to be a plurality of control units which can be parameterized by the operator's input.

The control unit reserves standard values for various settings in the sense of the present invention or determines them dynamically, as proposed in the course of the treatment or during a medical treatment on the output device. This may be performed optically by display on a display screen and/or acoustically by voice output via a loudspeaker.

Thus a standard value which has proven successful for a variety of treatment situations has been displayed for the operator. This standard value is the first parameter value characterizing the machine.

To enable this proposed parameter value for transfer to the control unit, where it parameterizes a corresponding program, or to enter a differing value, according to the invention, the operator must enter the numerical value of the first parameter into the input device.

This is preferably done by handwriting, in that the operator writes on a touch-sensitive surface, often in the form of a touchscreen display. This is done manually, for example, using a finger or a stylus.

This operator input is analyzed by a handwriting recognition software which is installed in the medical device and with which those skilled in the art are familiar and then it is translated into a numerical value. This numerical value derived from the operating input is the second parameter value characterizing the device.

Alternatively, such an operator input may also be made orally in that the operator makes the input as spoken words using a microphone. Speech recognition software, which is also installed and with which those skilled in the art are familiar, the translates this input into the second parameter value characterizing the device by a procedure similar to that used with the handwriting recognition software.

Input of the second parameter value characterizing the device is thus accomplished by handwriting or orally or optionally both.

The control unit compares the first and second parameter values characterizing the device and causes the first parameter value to be accepted by a control unit, which controls the device for parameterizing the corresponding control program for the case when the first and second parameter values characterizing the device match, and causes the first parameter value to be accepted or acceptable.

The transfer may thus be accomplished without further action on the part of the operator and may be effective immediately or it may occur by an additional operator input, for example, by operating a corresponding button (for example, as the operating surface of the touchscreen). This gives the operator an opportunity one more time to consider the parameter value that was just proposed.

The control unit can also verify whether the second parameter value is within certain limit values. If the second parameter value is outside of these limit values, the control unit can order neither the first parameter value nor the second to be transferred or transferable for parameterizing the corresponding control program.

In the sense of the invention, the control unit that performs the comparison and/or the verification and the transfer may be the same control unit, but different control units may also belong to the medical device, for example, in the event that a plurality of different control units are provided in the medical device.

The invention is thus based on the idea of prompting the operator of a medical device to reconsider the parameter value suggested by the device entering this parameter value or another parameter value of the corresponding parameter by handwriting or orally by the operator into the device instead of a simple confirmation, which can be performed in a simple and frivolous manner as in the past by touching a corresponding operating surface, for example.

### BRIEF DESCRIPTION OF THE FIGURES

Additional details and advantages of the invention are described in greater detail on the basis of exemplary embodiments depicted in the drawings, in which:

FIG. 1 shows a medical device according to the invention implemented as a dialysis machine for example;

FIG. 2 shows the screen contents of a medical device according to the state of the art;

FIG. 3 shows an embodiment of the screen contents of a medical device according to the invention;

FIG. 4 shows two other embodiments of the screen contents of a medical device according to the invention, and

FIG. 5 shows two other embodiments of the screen contents of a medical device according to the invention.

### DETAILED DESCRIPTION OF THE FIGURES

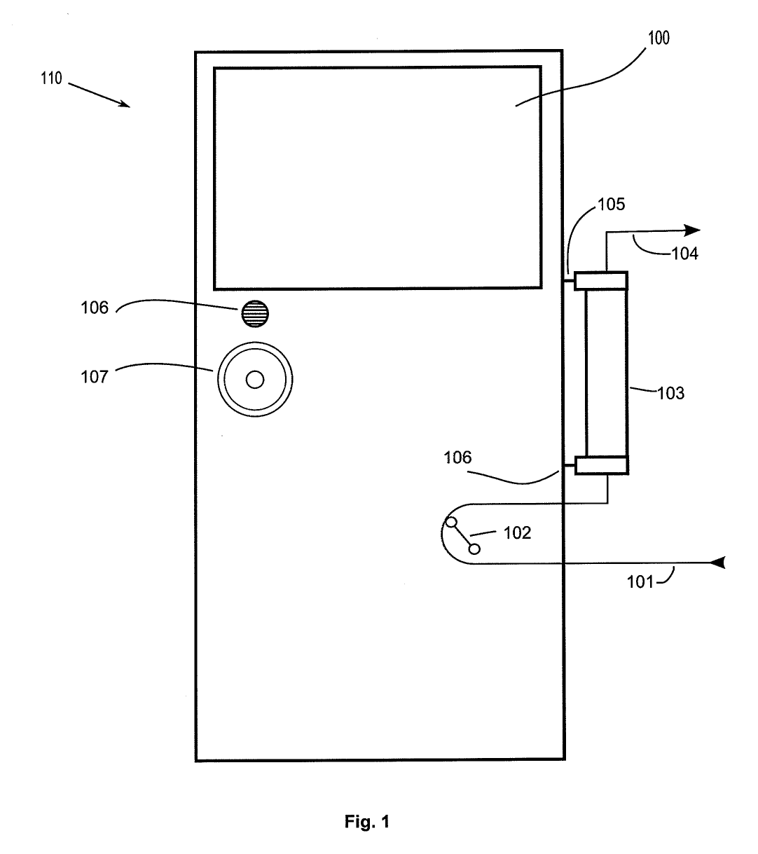

FIG. 1 shows schematically one embodiment of a medical device 110 according to the invention as a hemodialysis machine having a touchscreen display 100. The dialysis machine 110 shows parts of an extracorporeal blood circulation, which is just indicated, with an arterial bloodline 101 which draws the blood of a patient (not shown). The blood pump 102 conveys the blood through a dialysis filter 103, which is equipped with a semipermeable membrane that divides the extracorporeal blood circulation from a dialysis circulation in a semipermeable manner. The treated blood is returned to the patient through the venous line 104. Dialysate is pumped through the dialysis lines 105 and 106 through the dialysis filter 103, where it enters into a diffusive mass exchange with the patient's blood through the semipermeable membrane of the dialysis filter 103. If a pressure gradient is also established from the blood side of the dialysis filter to the dialysate side of the patient, plasma water is forced from the blood into the dialysate. Water can thus be withdrawn from the patient's blood. The dialysate is prepared in the hemodialysis machine 110 and is discarded after use. The hemodialysis machine 110 is also equipped with a loudspeaker 107 and a microphone 108 as additional input and output devices. It is clear to those skilled in the art that a loudspeaker can fundamentally also be used as a microphone because it can operate according to the same technical principle.

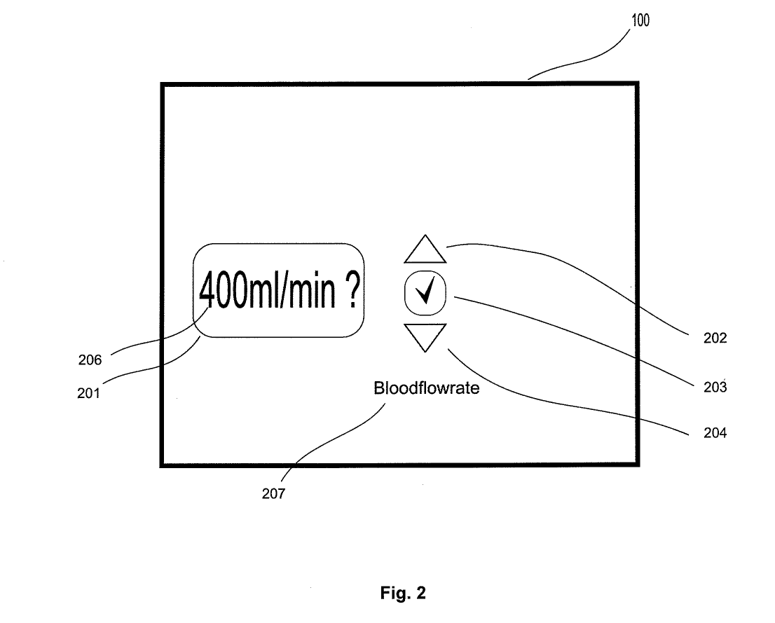

FIG. 2 shows a screen content of the touchscreen display 100 which is typical for the dialysis machine according to the state of the art. A first parameter value 206 characterizing the device, shown here as the numerical value “400” with the following unit “mL/min,” appears in the field 201. The question mark also shown in the field 201 characterizes the parameter as a suggestion that has not yet been accepted. The respective parameter 207 (blood flow rate) is also displayed.

At the right of the outlined area 201, three areas are available for operator input. To confirm the proposed parameter value 206, the operator must confirm it on the operator area 203, which is characterized by a checkmark with a border. The triangular areas 202 and 204, with which the parameter value 206 can be increased or decreased, are used to modify the proposed parameter value 206.

This operating concept illustrated in FIG. 2 entails the risk that the operator may accept a proposed standard value too frivolously, because only a few operating steps, namely only one step in the present example, are required to accept it.

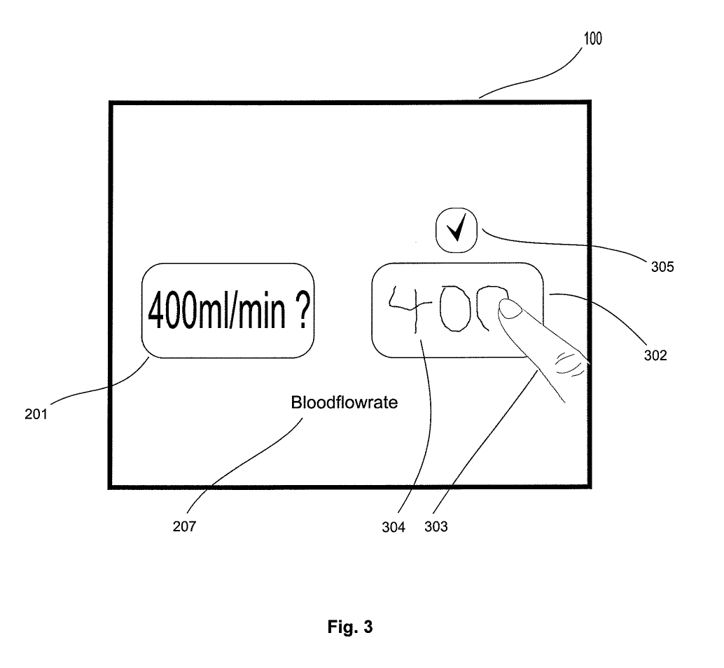

However, FIG. 3 illustrates an embodiment of the screen contents of a medical device according to the invention for the same step, such as that performed according to the state of the art in FIG. 2, in preparation of a medical device.

Here again, a standard value 206 is suggested by the control unit of the medical device. In contrast with FIG. 1, however, the operator cannot simply confirm this value but instead enters his own second parameter value 304 by hand in a field 302 which is provided for this purpose.

This is done by writing with one's finger 303 on a touch-sensitive surface which is embodied as a touchscreen display in FIG. 2.

Contact of the finger within the field 302 is recorded by a control unit and is displayed as a graphic display representing the parameter value 304.

If the operator has terminated the input of the second parameter value 304, an operating surface 305 may be available, such that when it is operated, a control unit is notified that the operator input is complete. An available program for recognition of handwriting attempts to translate the input into a machine-readable numerical value thereafter.

If the numerical value transmitted by the program for recognition of handwriting to a control unit matches the first parameter value 206, the control unit may be prompted that first parameter value is or may be accepted for further processing in a control unit that controls the machine.

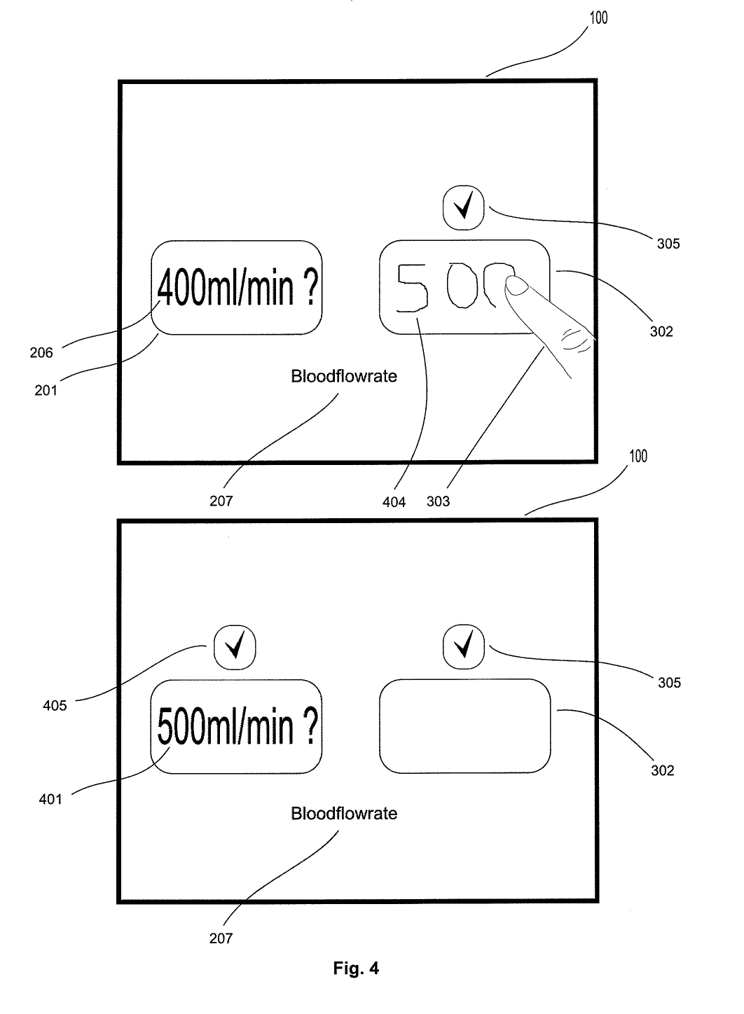

This may be accomplished with or without an act by the operator in that the operator operates a second operating area 405, for example, for confirmation (FIG. 4).

It is possible to provide that after the verification of whether the numerical value which was transmitted by the program for recognition of handwriting to a control unit matches the first parameter value 206, at least one acoustic, haptic or optical signal is output, such that its perceptible property depends on the result of the verification. For example, the matching of the first and second parameter values may be indicated by an acoustic tone, by a characteristic vibration of a touchscreen display equipped for this purpose or by a change in the color of the field 201. Similarly, if the first and second parameter values do not match, a different tone may be output, a different vibration of a touchscreen display equipped for this purpose may be prompted or the field 201 may be displayed in a different color than in the first case. In the sense of the invention, the output of a value or a warning or a symbol on the display 100, which depends on the result of the verification, is also an optical signal. The output of an acoustic, haptic or optical signal may similarly also occur as a function of a verification of an instruction input by the operator, for example, by verifying whether the instruction that has been input is implementable.

In the same way, acoustic, haptic and/or optical signals may be prompted, whose perceptible properties depend on the result of a verification of whether the second parameter value that has been input is within limit values.

FIG. 4 illustrates the situation when the second parameter value 404 that has been entered deviates from the suggestion 206 of the control unit. In the upper image in FIG. 4 the operator enters the deviating value 404 (“500”) instead of the proposed parameter value 206 (“400”).

As already described in conjunction with FIG. 3, a program for recognition of handwriting translates the operator's input into a machine-readable numerical value 401 and displays it in the field 201. At the same time, the field for input by the operator 302 may be cleared of the display of the handwriting representing the parameter value 404 in order to permit a renewed entry in field 302.

The operator now has the option of confirming the new value 401 recognized from his original input by confirming the corresponding field 405 or to repeat his input and to confirm the field 305 in the manner already described in conjunction with FIG. 3 after repeated input.

A numerical value entered by the operator and confirmed via the field 405 may then be accepted as the second parameter value by a control unit, which controls the machine.

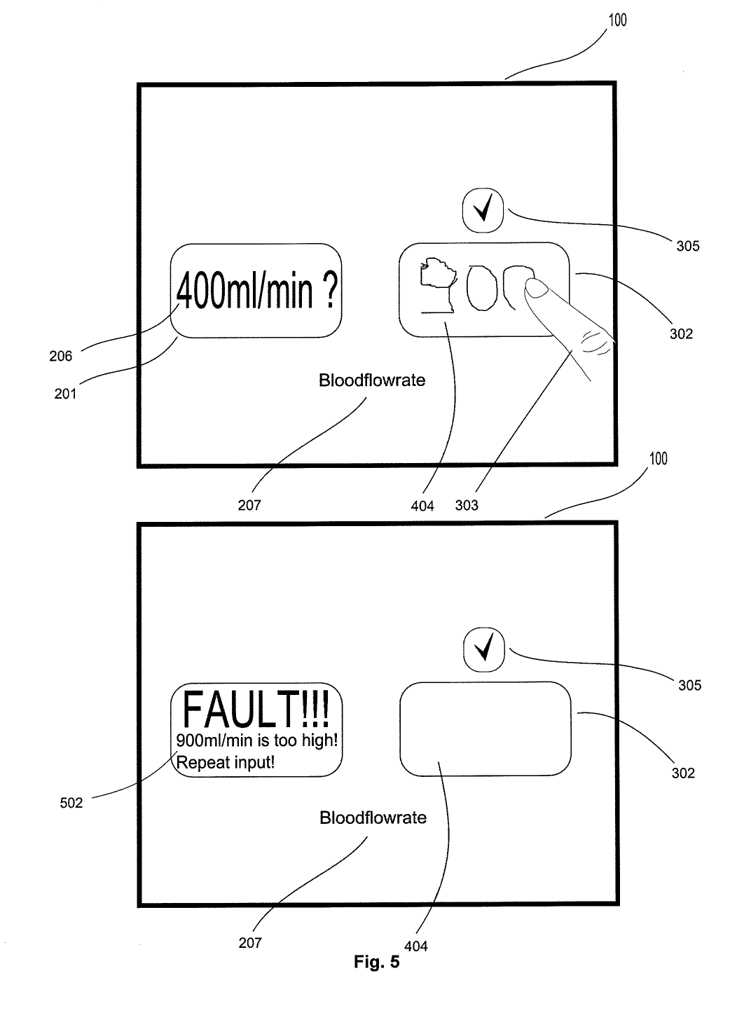

FIG. 5 shows two other embodiments of the screen contents of a medical device according to the invention for the situation when the numerical value entered by the user is outside of a range known to the control unit.

In the upper diagram in FIG. 5, the control unit proposes the numerical value “400” as the first parameter value 206, but the operator in deviation from that enters a second parameter value having the numerical value “900” in the field 302.

The control unit may be equipped so that any input by the operator is verified for plausibility and whether it complies with previously programmed limit values. Thus, for example, parameter 207, whose value is entered into FIG. 5, may be the blood flood rate in the extracorporeal blood circulation as shown in FIGS. 2 to 5.

The control unit can compare the parameter value thereby input with limit values programmed previously for the treatment and for the respective patient and can output a warning 502 (in the lower diagram in FIG. 5) when the parameter value input is outside of the limit values.

For example, a blood flow rate up to max. 700 mL/min is typical for a hemodialysis treatment of an adult. Different limit values apply to children. These limit values may be stored individually and are available to the control unit for each treatment.

In the case of such a violation of the limit value, the input area 302 may be deleted, as shown at the bottom of FIG. 5, and a renewed entry may be requested.

In addition, it is possible to verify whether a second parameter value that has been entered will result in failure to comply with therapeutic goals if this effect were to be achieved in the machine. The attending physician will often prescribe, on the basis of the treatment parameters, a certain period of time during which the patient should undergo dialysis in order to achieve a certain therapeutic goal.

However, certain situations may occur during the treatment, for example, cardiovascular fluctuations in the patient, necessitating a deviation from the original doctor's prescription. For example, the dialysate flow rate may be reduced to prevent the removal of certain substances from the blood too rapidly.

A reduced dialysate flow rate will result in the original therapeutic goal not being reached if the dialysis time is not lengthened. Such a deviation may be output to the operator as a warning via a display device. Like the message 502 in FIG. 5, this may indicate that the parameter value entered by the operator will lead to noncompliance with the doctor's prescription if it is implemented in the medical device. Such a message directed to the operator of the medical device, which is embodied here as a dialysis machine for example, may be formulated as follows for example:

“The new dialysis flow rate of 300 mL/min, which you have entered, will have the result, as long as the dialysis time remains unchanged, that the desired therapeutic goal will not be achieved. Should the dialysis time be lengthened by 1 hour to achieve the original therapeutic goal? Please enter ‘yes’, ‘no’ or ‘terminate’.”

The operator then enters a word as an instruction instead of a number in order to adjust the dialysis time based on the above message (enter “yes”), to leave the dialysis time unchanged (enter “no”) or to terminate the process (enter “terminate”). Then the control unit of the medical device verifies whether the operator's entry corresponds to an instruction that can be implemented and accepts the corresponding instruction depending on the result of this verification.

In addition, situations may occur during the treatment in which the operator would like to terminate the treatment prematurely. In such a case before the medical device actually terminates the treatment, it may inform the operator of the result of termination of the treatment by outputting a corresponding message on a display device and waiting for confirmation by operator input.

The result of termination of a treatment is often a failure to comply with the doctor's prescription.

A typical prescription for a dialysis treatment is the patient's weight, which is to be achieved because the patient can no longer adequately eliminate water ingested with his food because of his renal failure. This water accumulates in the blood and tissues of the patient. Consequently, at the start of the treatment, the patient has a body weight higher than that of a comparable patient with normal renal activity. One goal of dialysis is to remove the excess water from the patient's blood. The amount of water removed corresponds to the patient's weight loss during dialysis. Therefore a dialysis physician will often prescribe a final weight to be achieve, also known as the dry weight or target weight.

If the operator prematurely terminates the treatment, this can result in not reaching the prescribed weight of the patient.

To inform the operator about the consequences of terminating the treatment, the dialysis machine may output a message. This message may contain keywords.

These keywords may include, for example, “weight” or “continue.” The keywords characterize the consequences corresponding to the meaning of the word. Such a report might be as follows:

“If the treatment is terminated immediately, the prescribed WEIGHT will not be reached. For confirmation, please enter WEIGHT. If the treatment is to be continue, please enter CONTINUE.”

To confirm the termination, the operator should enter the keyword “WEIGHT” in an input device in the manner already described; if the keyword “CONTINUE” is entered the treatment is continued.

By entering the keyword, the operator is thus again given an opportunity to reconsider the consequences of his actions. This is the case in particular because the keyword to be entered characterizes the consequence of the input. This reduces the risk of rushed and unreflected actions. This pertains not only to the premature termination of a treatment initiated by an operator but also to any operator intervention.

The handwritten or oral input of a keyword is the input of an instruction in the sense of the invention.

In the case of entries by the operator, in which the installed program for recognition of handwriting cannot recognize a numerical value or an instruction, for example, due to an incomplete or illegible handwritten entry, a warning may be output and the operator may be instructed to repeat the input.

It is also conceivable that the program installed for recognition of handwriting will recognize the person making the entry on the basis of the handwriting entered and/or will recognize that the person making the entry is not among the persons allowed to perform entries.

For example, it may be provided that only certain persons such a physicians or attending medical personnel may be allowed to make entries into the medical device. The characteristics of the handwriting of these persons can be stored. The installed program for recognition of handwriting can in this way recognize which person provided for this purpose is making entries or whether a person not intended for this purpose is making entries.

In this way a protocol may be created and stored electronically to recognize which person has made entries into the medical device or it is possible to prevent unauthorized persons from making entries into the medical device by refusing to accept entries by unauthorized persons.

The handwritten operator entry described in FIGS. 3 through 5 may be supplemented or replaced by an oral operator entry in that as an alternative in addition to the optical output of proposed values, these values are output acoustically via speech output via loudspeaker 107 and alternatively or in addition to the handwritten operator input, these values are entered acoustically through spoken numerical values via a microphone 108.

The output of the proposed parameter values of certain first parameters can be supported by naming the parameter belonging to the first parameter value. Thus in the case of the blood flow rate as a parameter, the words “blood flow rate” may be output via the loudspeaker and linked to a following value, for example, “400.”

The operator input can then be accomplished in that the operator speaks the second parameter value or instructions out loud. A machine-readable number or an instruction can be determined from the spoken entry via the installed microphone 108 and a corresponding program. When a number is the second parameter value, it is possible to proceed in the same way as with handwritten input of the second parameter value.

This also relates in particular to the cases in which the oral input is not understandable, wherein the control unit can acoustically request the input to be repeated or when the second parameter value is outside of limit values, the control unit may announce a warning. Likewise, by analysis of the spoken word by analogy with handwriting recognition, it is possible to deduce the person speaking and consequently to prevent in the manner already described any entries from an unauthorized person from being implemented.

Furthermore, the combination of handwritten and oral input offers another possibility for increasing the safety of operator input. In this case the operator must perform a handwritten entry as well as an oral entry.

Handwritten and oral entries are verified separately and additionally for consistency of the two entries in the manner already described. Only if the handwritten entry and the oral entry are the same can the control unit allow the parameter value thereby entered to be forwarded.

With the help of the present invention, the work of the attending medical personnel in operation is supported on the one hand but on the other hand this also ensures that inappropriate settings are not made inadvertently or accidentally for the concrete treatment situation.

The embodiments are to be understood as examples and it is suggested to those skilled in the art that these embodiments be applied to other embodiments not shown here without any further inventive contribution.

1. A method for user input into a medical device comprising the steps
output of a first parameter value characterizing the medical device or a message directed at the operator of the medical device, handwritten or oral input of a second parameter value characterizing the device or an instruction,
verification of the second parameter or the instruction,
depending on this verification, ordering that the first or the second parameter value or neither the first nor the second parameter value is or can be accepted for further processing in a control unit which controls the device or that the instruction is accepted into a control unit which controls the device.

2. The method according to claim 1, wherein the verification includes the fact that the second parameter value is verified for whether it matches the first parameter value or whether it is within certain limit values or whether the instruction is implementable.

3. The method according to claim 1, wherein the output is via a display or via a loudspeaker.

4. The method according to claim 1, wherein the input is made by handwriting on a touch-sensitive surface or orally via a microphone.

5. The method according to claim 1, wherein, after the verification, at least one acoustic, haptic or optical signal is output, its perceptible property depending on the result of the verification.

6. The method according to claim 1, wherein, based on the handwritten or oral input, the person making the handwritten or oral input is inferred.

7. A device consisting of a medical device with an input device, an output device and a control unit, which are equipped for output, on the output device, of a first parameter value characterizing the device or a message directed at the operator of the medical device to order it and to verify a second parameter value characterizing the device or an instruction entered by handwriting or orally via the input device and to accept or make acceptable the first or second parameter value or not to accept or make acceptable either the first or the second parameter value in the control unit of the fluid management machine depending on the result of the verification or to accept the instruction into a control unit which controls the device.

8. The device according to claim 7, wherein the control unit is equipped to verify, in the verification, whether the second parameter value matches the first parameter value or whether it is within certain limit values or whether the instruction can be implemented.

9. The device according to claim 7, wherein the output device is a display or a loudspeaker.

10. The device according to claim 7, wherein the input device has a touch-sensitive surface or is a microphone.

11. The device according to claim 7, wherein the control unit is equipped to order the output of an acoustic, haptic and/or optical signal after the verification, such that the perceptible property of said signal depends on the result of the verification.

12. The device according to claim 7, wherein the control unit is equipped to infer the person having made the handwritten or oral input based on the handwritten or oral input.

13. The device according to claim 7, wherein the medical fluid management machine is a blood treatment machine and is equipped in particular for hemodialysis, peritoneal dialysis or plasmapheresis.

14. A device consisting of a medical device with an input device, an output device and a control unit, which are equipped for output, on the output device, of a first parameter value characterizing the device or a message directed at the operator of the medical device to order it and to verify a second parameter value characterizing the device or an instruction entered by handwriting or orally via the input device and to accept or make acceptable the first or second parameter value or not to accept or make acceptable either the first or the second parameter value in the control unit of the fluid management machine depending on the result of the verification or to accept the instruction into a control unit which controls the device, said control unit being equipped for executing at least one method according to claim 1.
